(19) United States
(12) Patent Application Publication
(10) Pub. No.: US 2022/0184405 A1
(43) Pub. Date: Jun. 16, 2022

(54) SYSTEMS AND METHODS FOR LABELING DATA IN ACTIVE IMPLANTABLE MEDICAL DEVICE SYSTEMS

(71) Applicant: Advanced Neuromodulation Systems, Inc., Plano, TX (US)

(72) Inventors: Scott DeBates, Frisco, TX (US); Tucker Tomlinson, Sachse, TX (US); Andrew Csavoy, Dallas, TX (US); Yagna Pathak, Skokie, IL (US); Lonnie Smith, Aubrey, TX (US); James Nagle, Celina, TX (US)

(21) Appl. No.: 17/370,250

(22) Filed: Jul. 8, 2021

Related U.S. Application Data
(60) Provisional application No. 63/124,409, filed on Dec. 11, 2020.

(51) Int. Cl. A61N 1/372; A61N 1/36; G16H 80/00; G16H 40/67; G16H 20/40; G16H 10/60

(52) U.S. Cl. CPC A61N 1/37282; A61N 1/36128; G16H 10/60; G16H 40/67; G16H 20/40; G16H 80/00

(57) ABSTRACT

The present disclosure provides systems and methods for labeling data in an active implantable medical device system. The method includes capturing data associated with a remote therapy session between a patient device and a clinician device, prompting, via a user interface, a user to label the captured data in response to the user selecting, via the user interface, at least one of i) an image of a patient displayed on the user interface, ii) a programming setting displayed on the user interface, and iii) an affected body area displayed on the user interface, receiving, in response to the prompting, via the user interface, a user input associated with the captured data, generating, based on the user input, a label associated with the captured data, and storing the generated label in association with the captured data.

### CROSS-REFERENCE TO RELATED APPLICATIONS

This application claims priority to provisional application Ser. No. 63/124,409, filed Dec. 11, 2020, which is incorporated herein by reference in its entirety.

### A. FIELD OF THE DISCLOSURE

The present disclosure relates generally to active implantable medical device systems, and more particularly to labeling data in such systems.

### B. BACKGROUND ART

Implantable medical devices have changed how medical care is provided to patients having a variety of chronic illnesses and disorders. For example, implantable cardiac devices improve cardiac function in patients with heart disease by improving quality of life and reducing mortality rates. Further, types of implantable neurostimulators provide a reduction in pain for chronic pain patients and reduce motor difficulties in patients with Parkinson's disease and other movement disorders. In addition, a variety of other medical devices currently exist or are in development to treat other disorders in a wide range of patients.

Many implantable medical devices and other personal medical devices are programmed by a physician or other clinician to optimize the therapy provided by a respective device to an individual patient. The programming may occur using short-range communication links (e.g., inductive wireless telemetry) in an in-person or in-clinic setting.

However, remote patient therapy is a healthcare delivery method that aims to use technology to manage patient health outside of a traditional clinical setting. It is widely expected that remote patient care may increase access to care and decrease healthcare delivery costs.

Active implantable medical devices (AIMD) are a class of therapeutic and/or diagnostic devices that contain electronic systems that allow for controlled delivery of therapy, which may be electrical stimulation of tissue, drug delivery via pumps, monitoring of implanted sensors etc. In many cases, these devices have settings that are remotely configurable after implantation using a wireless communications technology such as RF radios, Bluetooth, or WiFi.

For such devices, regularly updating settings may be a part of normal clinical care and maintenance. Historically, updating of AIMD settings is accomplished during a clinic visit where the patient travels to the clinic of their clinician, who uses an external programming device to make a local wireless connection to the AIMD. However, as telehealth technology becomes more available, AIMD systems may enable clinicians to remotely access and adjust device settings, allowing for updating settings without the patient's physical presence. This provides the benefits of allowing clinicians to serve patients without exposing them to travel burden, or exposure risks that may be present in the clinic.

### BRIEF SUMMARY OF THE DISCLOSURE

In one embodiment, the present disclosure is directed to a method for labeling data in an active implantable medical device system. The method includes capturing data associated with a remote therapy session between a patient device and a clinician device, prompting, via a user interface, a user to label the captured data in response to the user selecting, via the user interface, at least one of i) an image of a patient displayed on the user interface, ii) a programming setting displayed on the user interface, and iii) an affected body area displayed on the user interface, receiving, in response to the prompting, via the user interface, a user input associated with the captured data, generating, based on the user input, a label associated with the captured data, and storing the generated label in association with the captured data.

In another embodiment, the present disclosure is directed to a computing device for labeling data in an active implantable medical device system. The computing device includes a memory device, and a processor communicatively coupled to the memory device. The processor is configured to capture data associated with a remote therapy session between a patient device and a clinician device, prompt, via a user interface, a user to label the captured data in response to the user selecting, via the user interface, at least one of i) an image of a patient displayed on the user interface, ii) a programming setting displayed on the user interface, and iii) an affected body area displayed on the user interface, receive, in response to the prompting, via the user interface, a user input associated with the captured data, generate, based on the user input, a label associated with the captured data, and store the generated label in association with the captured data in the memory device.

In yet another embodiment, the present disclosure is directed to non-transitory computer-readable media having computer-executable instructions thereon. When executed by a processor of a computing device, the instructions cause the processor of the computing device to capture data associated with a remote therapy session between a patient device and a clinician device, prompt, via a user interface, a user to label the captured data in response to the user selecting, via the user interface, at least one of i) an image of a patient displayed on the user interface, ii) a programming setting displayed on the user interface, and iii) an affected body area displayed on the user interface, receive, in response to the prompting, via the user interface, a user input associated with the captured data, generate, based on the user input, a label associated with the captured data, and store the generated label in association with the captured data.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a diagram of one embodiment of a network environment for implementing remote therapy sessions.

FIG. 2 shows one embodiment of a user interface that may be used within the network environment shown in FIG. 1.

FIG. 3 shows one embodiment of a user interface that may be used within the network environment shown in FIG. 1.

FIG. 4 shows one embodiment of a user interface that may be used within the network environment shown in FIG. 1.

FIG. 5 shows one embodiment of a user interface that may be used within the network environment shown in FIG. 1.

FIG. 6 shows one embodiment of a user interface that may be used within the network environment shown in FIG. 1.

FIG. 7 shows one embodiment of a user interface that may be used within the network environment shown in FIG. 1.

FIG. 8 shows one embodiment of a user interface that may be used within the network environment shown in FIG. 1.

FIG. 8 shows one embodiment of a user interface that may be used within the network environment shown in FIG. 1.

FIG. 10 is a block diagram of one embodiment of a computing device.

Corresponding reference characters indicate corresponding parts throughout the several views of the drawings.

### DETAILED DESCRIPTION OF THE DISCLOSURE

The present disclosure provides systems and methods for labeling data in an active implantable medical device system. The method includes capturing data associated with a remote therapy session between a patient device and a clinician device, and prompting, via a user interface, a user to label the captured data in response to the user selecting, via the user interface, at least one of i) an image of a patient displayed on the user interface, ii) a programming setting displayed on the user interface, and iii) an affected body area displayed on the user interface. The method further includes receiving, in response to the prompting, via the user interface, a user input associated with the captured data, generating, based on the user input, a label associated with the captured data, and storing the generated label in association with the captured data.

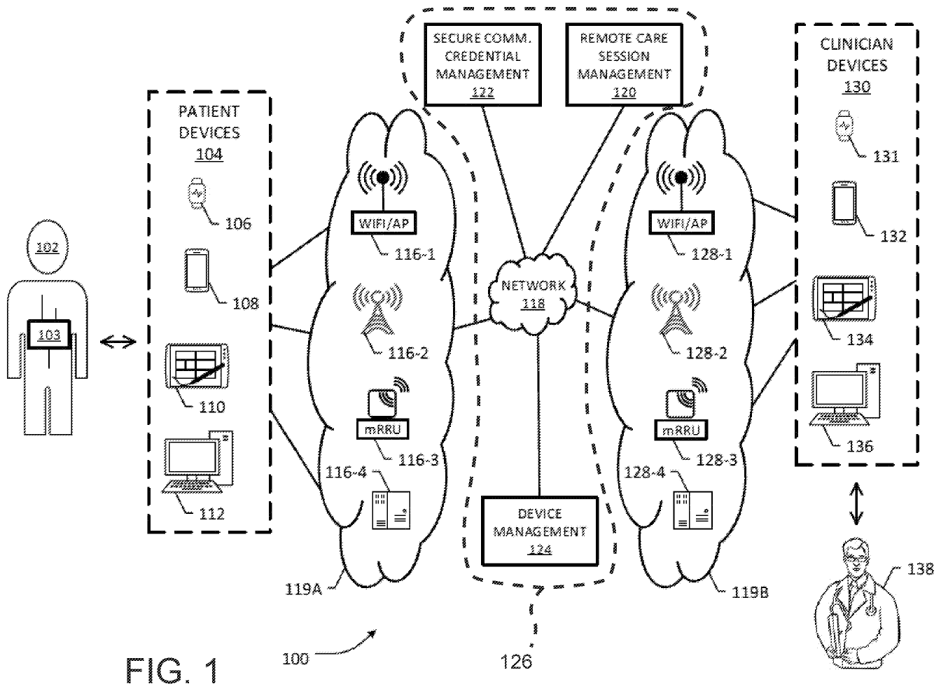

Referring now to the drawings, and in particular to FIG. 1, a network environment is indicated generally at 100. One or more embodiments of a remote care therapy application or service may be implemented in network environment 100, as described herein. In general, “remote care therapy” may involve any care, biomedical monitoring, or therapy that may be provided by a clinician, a medical professional or a healthcare provider, and/or their respective authorized agents (including digital/virtual assistants), with respect to a patient over a communications network while the patient and the clinician/provider are not in close proximity to each other (e.g., not engaged in an in-person office visit or consultation). Accordingly, in some embodiments, a remote care therapy application may form a telemedicine or a telehealth application or service that not only allows healthcare professionals to use electronic communications to evaluate, diagnose and treat patients remotely, thereby facilitating efficiency as well as scalability, but also provides patients with relatively quick and convenient access to diversified medical expertise that may be geographically distributed over large areas or regions, via secure communications channels as described herein.

Network environment 100 may include any combination or sub-combination of a public packet-switched network infrastructure (e.g., the Internet or worldwide web, also sometimes referred to as the “cloud”), private packet-switched network infrastructures such as Intranets and enterprise networks, health service provider network infrastructures, and the like, any of which may span or involve a variety of access networks, backhaul and core networks in an end-to-end network architecture arrangement between one or more patients, e.g., patient(s) 102, and one or more authorized clinicians, healthcare professionals, or agents thereof, e.g., generally represented as caregiver(s) or clinician(s) 138.

Example patient(s) 102, each having a suitable implantable device 103, may be provided with a variety of corresponding external devices for controlling, programming, otherwise (re)configuring the functionality of respective implantable medical device(s) 103, as is known in the art. Such external devices associated with patient(s) 102 are referred to herein as patient devices 104, and may include a variety of user equipment (UE) devices, tethered or untethered, that may be configured to engage in remote care therapy sessions. By way of example, patient devices 104 may include smartphones, tablets or phablets, laptops/desktops, handheld/palmtop computers, wearable devices such as smart glasses and smart watches, personal digital assistant (PDA) devices, smart digital assistant devices, etc., any of which may operate in association with one or more virtual assistants, smart home/office appliances, smart TVs, virtual reality (VR), mixed reality (MR) or augmented reality (AR) devices, and the like, which are generally exemplified by wearable device(s) 106, smartphone(s) 108, tablet(s)/phablet(s) 110 and computer(s) 112. As such, patient devices 104 may include various types of communications circuitry or interfaces to effectuate wired or wireless communications, short-range and long-range radio frequency (RF) communications, magnetic field communications, Bluetooth communications, etc., using any combination of technologies, protocols, and the like, with external networked elements and/or respective implantable medical devices 103 corresponding to patient(s) 102.

With respect to networked communications, patient devices 104 may be configured, independently or in association with one or more digital/virtual assistants, smart home/premises appliances and/or home networks, to effectuate mobile communications using technologies such as Global System for Mobile Communications (GSM) radio access network (GRAN) technology, Enhanced Data Rates for Global System for Mobile Communications (GSM) Evolution (EDGE) network (GERAN) technology, 4G Long Term Evolution (LTE) technology, Fixed Wireless technology, 5^{th} Generation Partnership Project (5GPP or 5G) technology, Integrated Digital Enhanced Network (IDEN) technology, WiMAX technology, various flavors of Code Division Multiple Access (CDMA) technology, heterogeneous access network technology, Universal Mobile Telecommunications System (UMTS) technology, Universal Terrestrial Radio Access Network (UTRAN) technology, All-IP Next Generation Network (NGN) technology, as well as technologies based on various flavors of IEEE 802.11 protocols (e.g., WiFi), and other access point (AP)-based technologies and microcell-based technologies such as femtocells, picocells, etc. Further, some embodiments of patient devices 104 may also include interface circuitry for effectuating network connectivity via satellite communications. Where tethered UE devices are provided as patient devices 104, networked communications may also involve broadband edge network infrastructures based on various flavors of Digital Subscriber Line (DSL) architectures and/or Data Over Cable Service Interface Specification (DOCSIS)-compliant Cable Modem Termination System (CMTS) network architectures (e.g., involving hybrid fiber-coaxial (HFC) physical connectivity). Accordingly, by way of illustration, an edge/access network portion 119A is exemplified with elements such as WiFi/AP node(s) 116-1, macro/microcell node(s) 116-2 and 116-3 (e.g., including micro remote radio units or RRUs, base stations, eNB nodes, etc.) and DSL/CMTS node(s) 116-4.

Similarly, clinicians 138 may be provided with a variety of external devices for controlling, programming, otherwise (re)configuring or providing therapy operations with respect to one or more patients 102 mediated via respective implantable medical device(s) 103, in a local therapy session and/or remote therapy session, depending on implementation and use case scenarios. External devices associated with clinicians 138, referred to herein as clinician devices 130, may include a variety of UE devices, tethered or untethered, similar to patient devices 104, which may be configured to engage in remote care therapy sessions as will be set forth in detail further below. Clinician devices 130 may therefore also include devices (which may operate in association with one or more virtual assistants, smart home/office appliances, VRAR virtual reality (VR) or augmented reality (AR) devices, and the like), generally exemplified by wearable device(s) 131, smartphone(s) 132, tablet(s)/phablet(s) 134 and computer(s) 136. Further, example clinician devices 130 may also include various types of network communications circuitry or interfaces similar to that of patient device 104, which may be configured to operate with a broad range of technologies as set forth above. Accordingly, an edge/access network portion 119B is exemplified as having elements such as WiFi/AP node(s) 128-1, macro/microcell node(s) 128-2 and 128-3 (e.g., including micro remote radio units or RRUs, base stations, eNB nodes, etc.) and DSL/CMTS node(s) 128-4. It should therefore be appreciated that edge/access network portions 119A, 119B may include all or any subset of wireless communication means, technologies and protocols for effectuating data communications with respect to an example embodiment of the systems and methods described herein.

In one arrangement, a plurality of network elements or nodes may be provided for facilitating a remote care therapy service involving one or more clinicians 138 and one or more patients 102, wherein such elements are hosted or otherwise operated by various stakeholders in a service deployment scenario depending on implementation (e.g., including one or more public clouds, private clouds, or any combination thereof). In one embodiment, a remote care session management node 120 is provided, and may be disposed as a cloud-based element coupled to network 118, that is operative in association with a secure communications credentials management node 122 and a device management node 124, to effectuate a trust-based communications overlay/tunneled infrastructure in network environment 100 whereby a clinician may advantageously engage in a remote care therapy session with a patient.

In the embodiments described herein, implantable medical device 103 may be any suitable medical device. For example, implantable medical device may be a neurostimulation device that generates electrical pulses and delivers the pulses to nervous tissue of a patient to treat a variety of disorders.

One category of neurostimulation systems is deep brain stimulation (DBS). In DBS, pulses of electrical current are delivered to target regions of a subject's brain, for example, for the treatment of movement and effective disorders such as PD and essential tremor. Another category of neurostimulation systems is spinal cord stimulation (SCS) for the treatment of chronic pain and similar disorders.

Neurostimulation systems generally include a pulse generator and one or more leads. A stimulation lead includes a lead body of insulative material that encloses wire conductors. The distal end of the stimulation lead includes multiple electrodes, or contacts, that intimately impinge upon patient tissue and are electrically coupled to the wire conductors. The proximal end of the lead body includes multiple terminals (also electrically coupled to the wire conductors) that are adapted to receive electrical pulses. In DBS systems, the distal end of the stimulation lead is implanted within the brain tissue to deliver the electrical pulses. The stimulation leads are then tunneled to another location within the patient's body to be electrically connected with a pulse generator or, alternatively, to an “extension.” The pulse generator is typically implanted in the patient within a subcutaneous pocket created during the implantation procedure.

The pulse generator is typically implemented using a metallic housing (or can) that encloses circuitry for generating the electrical stimulation pulses, control circuitry, communication circuitry, a rechargeable battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on the proximal end of a stimulation lead.

Although implantable medical device 103 is described in the context of a neurostimulation device herein, those of skill in the art will appreciate that implantable medical device 103 may be any type of implantable medical device. Further, although at least some of the examples provided herein relate to remote therapy sessions involving deep brain stimulation, those of skill in the art will appreciate that the embodiments described herein are applicable to remote therapy sessions for patient with other implantable devices (e.g., neurostimulators for chronic pain, or drug delivery pumps).

In systems including an active implantable medical device (AIMD), such as network environment 100 (shown in FIG. 1), there is an opportunity to collect longitudinal data associated with the adjustments in settings and overall therapy. However, one challenge with analyzing collected data is labeling the data in a meaningful way, such that end users or machine systems can effectively parse the data. For instance, a system might collect one hundred hours of video data to capture ten discrete events. If those events are labeled, the task of finding and processing the video of the events is dramatically more efficient than if the entire one hundred hours must be processed.

The systems and methods described herein provide two related data labeling approaches. The approaches may be implemented, for example, within network environment 100 (shown in FIG. 1). The first approach involves treating specific collections of AIMD settings as data points, and labeling those data points with labels that indicate something about the quality of the settings, or something about the context of those settings. The second labeling approach involves labeling of associated data collected either by the AIMD system directly, or by external sensors linked to the system. These may be general behavior sensors such as accelerometers which might reflect behavioral consequences of changes in therapy, or physiologic sensors which might reflect the direct response to the AIMD such as heart rate for pacemakers, local field potentials or neural spiking for neurostimulators, or blood glucose for insulin pumps. While the advent of connected AIMD systems provides a specific extended use case for labeling of data, the elements of this disclosure are also applicable to AIMD systems that do not include network connectivity. This is especially relevant as increases in the computing power of mobile devices, especially with regards to machine learning, provide extended utility to data collection and labeling even in cases where the system is isolated. The labels generated using the systems and methods described herein may be used, for example, for training purposes, for data analysis purposes, for diagnostic purposes, etc.

As used herein, a ‘program’ for an AIMD refers to a collection of settings that defines operating behavior of the AIMD. An AIMD may maintain several programs, allowing the user to switch between different modes of behavior in order to obtain different therapeutic effects.

Labeling AIMD program settings data (i.e., the first approach noted above) using the systems and methods described herein provides several distinct benefits. The history of labeled programs may be presented to the user so that the user can evaluate the efficacy of settings and identify important trends. Alternatively, labels enable machine learning algorithms, or algorithmic clustering systems to predictively identify settings that might result in similar labels. This is particularly useful in cases where the label indicates some rating or efficacy of the therapy associated with the program settings.

For instance, if a certain range of settings results in a specific side effect, similar settings may result in a similar side effect. Predicting the labels before application of the settings changes allows the system and user to effectively anticipate the outcome of some combinations of settings, improving efficiency and efficacy in identifying optimal therapy settings. In some embodiment, an automated response may be generated, such as notifying the patient or clinician via a connected external programming device, or notifying the patient of clinician via a network messaging system such as email or SMS.

Presentation of longitudinal labels also facilitates identifying trends in the data. This may be useful, for example, where a response to the AIMD settings is expected to change over time, as in the case of AIMD used to treat progressive disorders. In progressive medical conditions, symptoms are expected to worsen or change with time. Tracking changes in the labels associated with similar settings overtime allows the user or an automated system to assess trends in the labeled feature.

The assessment may include, for example, a rating of efficacy in symptom suppression, changes in the area or extent of the body covered by therapy, rating of the severity and extent of side effects, or a rating of patient preference. Analysis of this type of trend allows the clinician to more effectively evaluate both the status of the pathology, and the efficacy of the therapy provided by the AIMD. This analysis can be performed at multiple levels or scales within a single programming session or fixed time period to show discrete improvement or change, or across multiple programming sessions or a long time period to track trends in therapy or pathology, and across a population to assess whether factors such as changes in clinical strategy, medication availability or access have altered the prevalence of event occurrence.

Labeling of associated data (i.e., the second approach noted above) using the systems and methods described herein also provides several distinct benefits. Associated data is often collected continuously, making the task of isolating specific event times consuming, and potentially labor intensive. If sufficient labeled data is available, algorithmic or machine learning approaches can be applied to establish an automated system which identifies events of interest in real time. Labeling of associated data in this manner may also provide a label for the associated program settings.

For instance, a wrist mounted accelerometer may allow for the detection of an increase in dyskinesia resulting from a change in settings of a neurostimulator. A label of ‘dyskinesia’ could be applied both to the accelerometer data, and to the settings that gave rise to the dyskinesia. Similarly, the output of clinical scales assessed at the same time the associated data was collected may also be used to label the data.

Validated clinical scales are often used to assess severity of symptoms. Some examples of clinical scales that might be used this way include the Visual Analog Scale (VAS) for pain assessment, the Unified Parkinson's Disease Rating Scale (UPDRS), the Unified Dystonia Rating Scale (UDRS), etc. Aggregation of data from sensors with labels enables users and, in particular, automated machine systems to learn how to identify data associated with the labeled states. This provides the capability for the user or automated machine system to detect onset of potentially harmful symptoms or side effects and respond appropriately.

An automated response may include notifying the patient or clinician via a connected external programming device, notifying the patient or clinician via a network message such as email or SMS, or automatic adjustment of AIMD settings using a feedback control system. In response to a notification, the patient may seek clinician assistance, or adjust the AIMD settings directly depending on the level of available control and the detected issue.

Additionally, similar to labeling of program data, labeling of associated data allows for the assessment of trends. For example, a wrist mounted accelerometer on a Parkinson's disease patient that is labeled with UPDRS scores might provide a longitudinal assessment of stability or progression of tremor or dyskinesia symptoms. This analysis can be performed at multiple levels or scales within a single programming session or fixed time period to show discrete improvement or change, or across multiple programming sessions or a long time period to track trends in therapy or pathology, and across a population to assess whether factors such as changes in clinical strategy, medication availability or access have altered the prevalence of event occurrence. Further, in cases where data is labeled with a rating or score, this methodology enables comparison of the score to the population to determine the difference between individual efficacy as compared to the expected efficacy.

Data on program settings or from associated sensors are typically available continuously. However, data may be collected and labeling may be applied with a number of temporal schema. The data collection and labeling may be implemented, for example, using clinician device 130. Alternatively, the embodiments described herein may be implemented using any suitable computing device, including other devices within network environment 100.

In one example of an explicit sampling schema, a user (e.g., clinician 138) deliberately selects a datum to label, triggering the system to log the datum and the label simultaneously. This schema may be used, for example, in scenarios where clinician 138 is evaluating changes in therapy settings, and enters a label (e.g., using a user interface on clinician device 130) indicating efficacy of a particular program setting. Entering the label triggers the system to log the specific settings along with the label. This has the advantage of only storing data when a label is generated, but does not store comparison data at times when no label is generated. Other examples of explicitly sampled data include the presentation of a clinical test, such as the VAS for a spinal cord neurostimulator patient, or a spiral drawing test for an essential tremor patient with a Deep Brain Stimulation System.

Alternatively, an implicit labeling schema may be used, in which the AIMD system monitors the actions of a user (e.g., patient 102 and/or clinician 138), and automatically apply labels based on the monitored user activity.

In one example, microphones built into an external programming device (e.g., clinician device 130) are used to monitor the speech of the user, and to flag certain keywords (e.g., “good”, “bad”, “side-effect”, etc.) and apply those keywords as labels.

Another example applies in testing scenarios where a user is slowly increasing or decreasing a parameter setting to evaluate the impact of that setting. In this situation, the system may detect when the user either stops changing the setting, stops the AIMD output (e.g., stops applied stimulation), or reverses the last change in the setting. This would allow the AIMD system to apply a label indicating that the final setting was an identified limit which clinician 138 had elected not to go beyond. This implicit schema advantageously continues to label data even when the user is not explicitly entering labels, at the potential expense of specificity and accuracy.

Alternatively, the system may log continuous data, in the case of sensors sampling at a regular frequency, or in the case of AIMD program settings sampling every time settings are changed. The user may then review the logged data at a later date and add labels as they deem appropriate. This schema is particularly advantageous for scenarios where the event of interest is rare, and data must be sampled for a long period to capture events of interest, or cases where a long period of data is necessary to make an adequate evaluation for the label. For example, in one embodiment, clinician 138 may review stored video and add one or more labels to the stored video. The labels may be appended, for example, to a session log including the stored video.

In another embodiment, data collection is event triggered. This is somewhat similar to the explicit sampling scheme. However, in this scenario, the data point is only logged if a condition associated with the label is detected. This detection may be made either by a user, or by an automated system. If events are detected by an automated system, that system can additionally notify clinician 138 and/or patient 102 for confirmation of the label. This allows the system to improve the fidelity of the labels, and to apply adaptive learning, or continuous update algorithms to improve event detection.

A specific non-obvious event case may correspond to the entry of a different label. For example, the user may choose to explicitly label some program settings as ineffective. This event could trigger the AIMD system to use logs of how long the program was active at those settings to label the data with an indicator of how long the settings were tested. This specific example would allow for future assessment of how reliable the efficacy label might be.

In the example sampling schemes described herein (explicit, continuous, event triggered), the label entry is associated temporally with the data being labeled. In cases where the label is entered on the same device that is sampling the data (e.g., when using explicit sampling to labeling active program settings on an external programming device, or when reviewing continuous data to apply labels), the time of the label can be simply derived as the timestamp of the current datum. In the case of data that is not collected by the same device, synchronization issues may occur.

In general the synchronization between the timekeeping on the label entry device and on the data logging device must be close enough to accurately and precisely confirm which datum the label applies to. For coarse labels that may apply to a long sequence of datum, such as labeling of an entire video sequence as “walking,” devices may be simply synchronized by one device sending the other device its current time, and computing the offset. For more precise labeling, the latency of network communication becomes an issue, and more advanced synchronization techniques may be applied, such as the Network Time Protocol, Precision Time Protocol, or synchronization to GPS time signals, which can generally reduce the offset between device clocks to milliseconds or less.

The method of label entry is important to success of labeling systems, as the utility of labeled data relies on access to a history of accurately labeled data in order to present trends, or train automatic systems. In the case of explicit or event driven labeling, the user can often be relied upon to have access to the external programming device. Therefore, several methods of data collecting may focus on interfaces that rely upon the user having access to an external programming device (e.g., clinician device 130). In this case, the external programming device is presumed to be a mobile device with a touch screen such as a tablet or phone. These interfaces may be replicated or extended on other devices, such as desktop computers or web applications, to address labeling of continuous data, or labeling in cases where the user is monitoring data using an alternative device such as a desktop or laptop computer.

User interfaces with a screen enable for the user to directly select a user interface (UI) element (e.g., an icon, an image, displayed text, etc.) for which they want to add a label. This could manifest as a long press on the UI element. UI elements amenable to this sort of access include video windows to label video contents, and UI elements associated with modifying the program settings. Additional labeling specificity can be created by noting the location within the UI element that was selected to enter the label. For instance, the user might select a portion of a video focused on a patient's legs to enter a label of gait disturbance, or might select a specific program setting control such as amplitude to enter a label for the program settings. An alternative method of entering labels is via fixed UI elements that are either embedded in the UI (which allows users to evaluate and modify settings), or via a menu, drawer, or other system to call up additional interfaces and options.

Once data is selected for labeling, the user may enter a label via several mechanisms. In one embodiment, the system may provide a list of potential labels which the user may select from. It is possible that labels may be grouped to facilitate labeling. For example, labeling how effective a certain program setting is might be combined with a label describing how long the clinician observed the setting to evaluate the efficacy, or data from an external video sensor might be labeled both with a tremor rating score, and a gait rating score. This list could be presented via a drop-down menu, a pop-up grid of options, a nested tree of labels, etc.

List items may be defined by the manufacturer and/or by the end user to provide more granular label detail. Alternatively, instead of a pre-defined list, label entry may utilize a free-form system. This might take the form of a speech-to-text system where the user simply states the label, a text box which allows entry of text via a keyboard, scanning of external text using a camera attached to the external programming device, and/or conversion of handwritten notes to characters via an Optical Character Recognition (OCR) software using a digital stylus or scanned from paper. Labels including a numeric value may be entered via a text box, a slider or scroll UI, or via any of the free-form methods above. Alternatively, labels may be automatically entered by the execution of certain tasks or events. For instance, if the AIMD system enables clinician 138 to present patient 102 with a standardized test, and log the results, the system may automatically apply the results as labels to the current program settings. Event triggered sample collection may also trigger the system to prompt the user to enter a label using either a list or free-form entry system.

The examples discussed above generally focus on labeling provided by clinician 138 in their role as the subject matter expert most qualified to provide labels for data. There are, however, scenarios where other users of AIMD systems may provide labels. Patient 102, for example, may provide a self-assessment of symptom status, which may be used as a label for settings or data collected between sessions with clinician 138. This data then forms a valuable report from which clinician 138 may make an informed assessment of the patient's therapy status and variability when not in the clinic. For example, clinician 138 may use a historical display of settings to note that symptoms are worse when patient 102 adjusts settings in a specific manner. For example, a DBS patient's dyskinesia may be worse when patent 102 increases stimulation amplitude. Patient 102 may enter data labels utilizing any of the mechanisms described herein in associated with clinician 138, including tapping or long pressing on video, graphic displays such as body maps, application controls or setting displays, and/or in explicit label entry interfaces.

As above, an automated system may parse speech of the user (e.g., patient 102) and automatically apply labels based on the content. For example, natural language processing could be used to detect when patient 102 makes assessments such as ‘better’ or ‘worse’, and to apply appropriate labels to the current settings, or to associated data. Such systems may include either simply listening for keywords, or parsing for more complex syntax, such as a symptom associated with an assessment (e.g., “my pain is worse” or “my tremor seems better”). As discussed for the clinician case, automated labeling improves the rate of label application at the potential expense of accuracy.

Many different types of data may be labeled using the systems and methods described herein. The following types of data are merely examples, and those of skill in the art will appreciate that any suitable type of data may be labeled using the systems and method described herein.

In one embodiment, for example, video data of patient 102 and/or clinician 138 captured during a telehealth session, or captured offline for submission to telehealth system may be labeled. For example, such data may be labeled by automatically or manually parsing movement from the video (upper and lower limbs, body, hands, head, face, feet, etc.). Further, the date, time, and/or location of the event occurrence may be labeled. Further, contextual labels may be generated and/or curated by patient 102 or clinician 138. Further, labels may be generated by automatically adding parameters of the active AIMD program. In another example, audio data of patient 102 and/or clinician 138 may be labeled during a telehealth session. Such data may be labeled by automatically parsing speech from the audio data.

Other types of data that may be labeled includes notes entered directly in the AIMD system by the user, notes entered either outside the AIMD system or entered in free-form and parsed with a natural language processing system, changes in AIMD settings made by patient 102 and/or clinician 138, changes in non-AIMD therapy entered by a user such as medication changes, and records of clinical tests such as spiral drawing or finger tapping tests either performed on a device connected to the AIMD system, or performed separately and imported into the AIMD system.

Further, other types of data that may be labeled include records from sensors such as accelerometers, HR sensors, BP sensors, RR sensors, PO2 sensors, and galvanic skin resistivity sensors, etc. linked to the AIMD system, logs from AIMD system devices such as the AIMD, external programming devices, connected cloud services etc., Time and duration of interactions during telehealth sessions, and location and relative movement data of patient 102 for a known period of time (e.g. min/max distances traveled from home, locations visited outside home (for example, gym, supermarket, work site, etc.)).

Many different types of labels may be applied using the system and methods described herein. For example, content labels may include behavioral contents of video data, or content labels associated with device settings. For device settings, labels may define how well tested particular settings are (e.g., untested, undocumented, tested within session observation, part of long term follow-up), may note side effects (e.g., dyskinesia, paresthesia, balance disturbance, vocal disturbance, muscle pulling, etc.), may note ocular disturbances (e.g., gaze deviation, diplopia, phosphenes, nystagmus, etc.), or may note an affected body area.

Therapy quality labels may include efficacy notes (e.g., ineffective, partially effective, totally effective), efficiency notes (e.g., power efficiency optimized, power efficiency unexplored), or patient self-assessments/impressions of their therapy state. Patient self-assessments/impressions may be entered directly by patient 102 (e.g., using patient device 104), and/or may be logged in a separate application, such as a symptom diary, and synchronized at a later time.

Further, context labels may include timing of data collection relative to events or activities (e.g., during walking, after standing, before medication), a location of patient 102 and/or clinician 138 (e.g., geolocation data or site of service (physician office, patient home, caregiver home, assisted living facility, managing clinician office, hospital clinic, etc.)), or a status of datum not included in the labeled data (e.g., medication status, recent meals, patient report of recent symptoms). In some embodiments, the status of datum not included in the labeled data may be directly captured by other systems. Further, label creation of this sort allows the user to effectively add arbitrary data to the log.

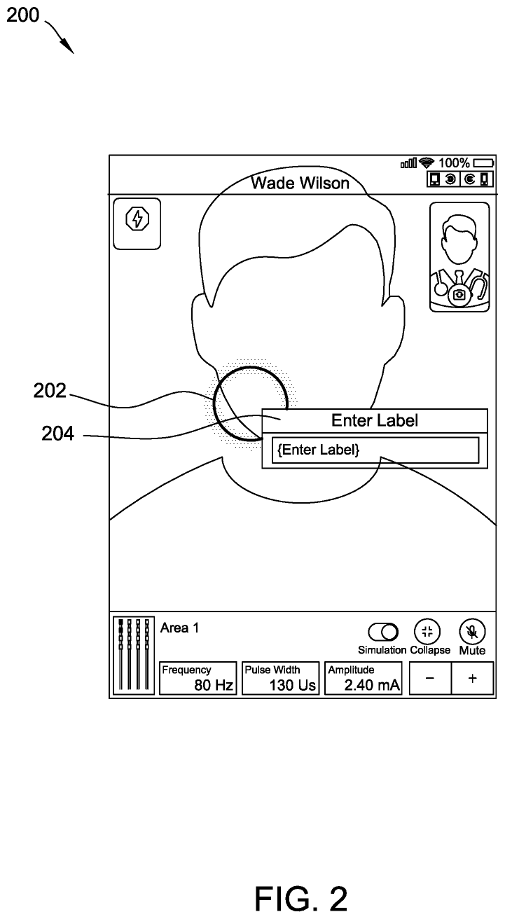

FIGS. 2-9 are example user interfaces that may be displayed, for example on patient device 104 or clinician device 130 (both shown in FIG. 1).

FIG. 2 shows one embodiment of a user interface 200 (e.g., to be displayed to clinician 138). User interface 200 enables a user (e.g., clinician 138) to modify settings of a neurostimulation system. Further, as shown in FIG. 2, the user has selected a facial area 202 (e.g., by making a long-press selection on user interface 200), enabling the user to enter a free-form label in a text box 204.

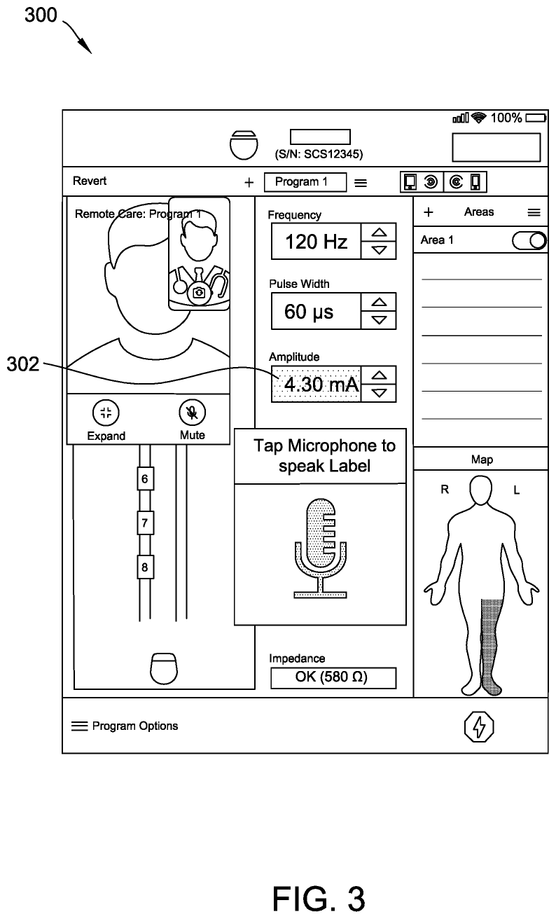

FIG. 3 shows another embodiment of a user interface 300 (e.g., to be displayed to clinician 138). User interface 300 enables a user (e.g., clinician 138) to modify settings of a neurostimulation system. Further, as shown in FIG. 3, the user has selected a programming setting 302 (e.g., by making a long-press selection on user interface 300), enabling the user to record a spoken label using a microphone on the device displaying user interface 300.

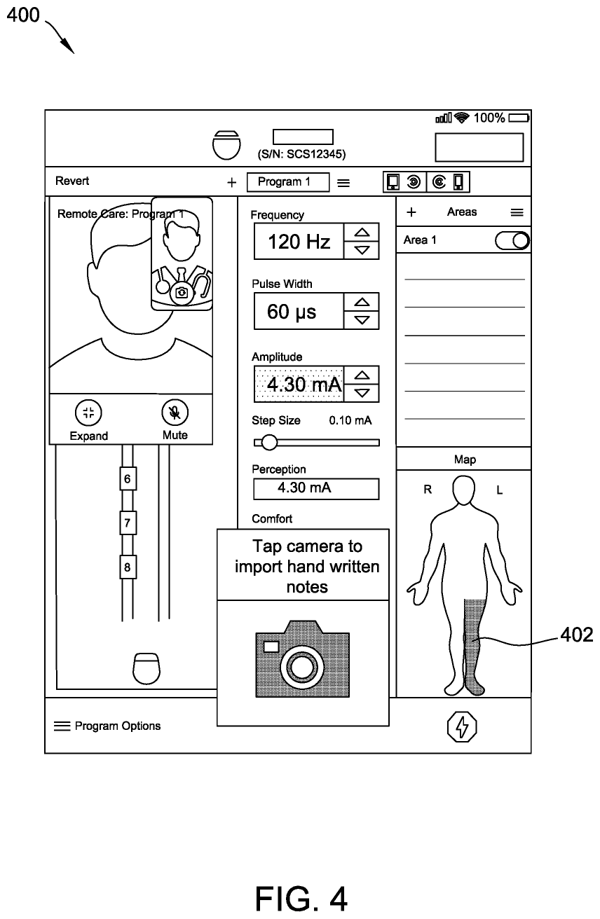

FIG. 4 shows another embodiment of a user interface 400 (e.g., to be displayed to clinician 138). User interface 400 enables a user (e.g., clinician 138) to modify settings of a neurostimulation system. Further, as shown in FIG. 4, the user has selected an affected body area 402 (e.g., by making a long-press selection on user interface 400), enabling the user to import a hand-written note as a label using a camera on the device displaying user interface 400.

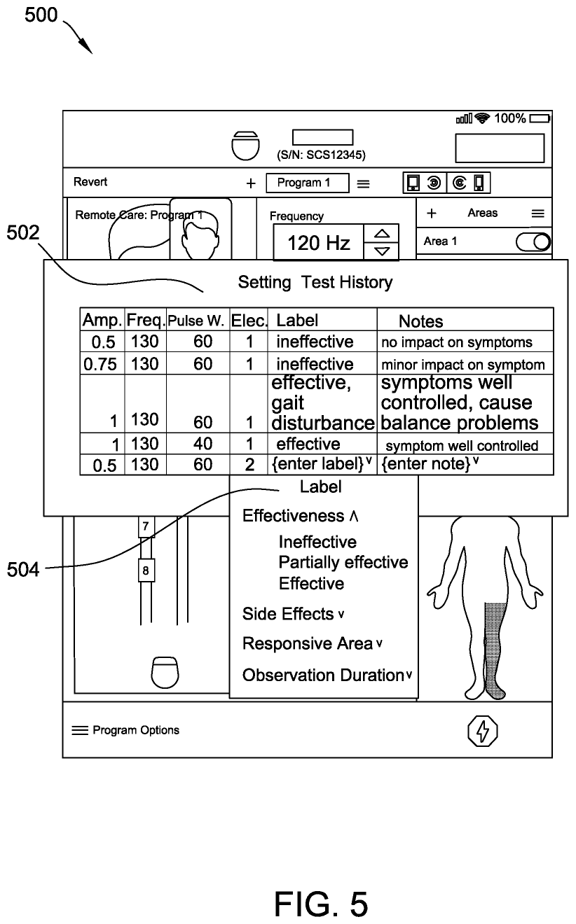

FIG. 5 shows another embodiment of a user interface 500 (e.g., to be displayed to clinician 138). User interface 500 enables a user (e.g., clinician 138) to modify settings of a neurostimulation system. Further, as shown in FIG. 5, user interface 500 shows a tabular history 502 of therapy settings with previously applied labels, and includes an interface 504 (here, a drop-down menu with a nested tree of label options) for adding new labels to the current stimulation settings.

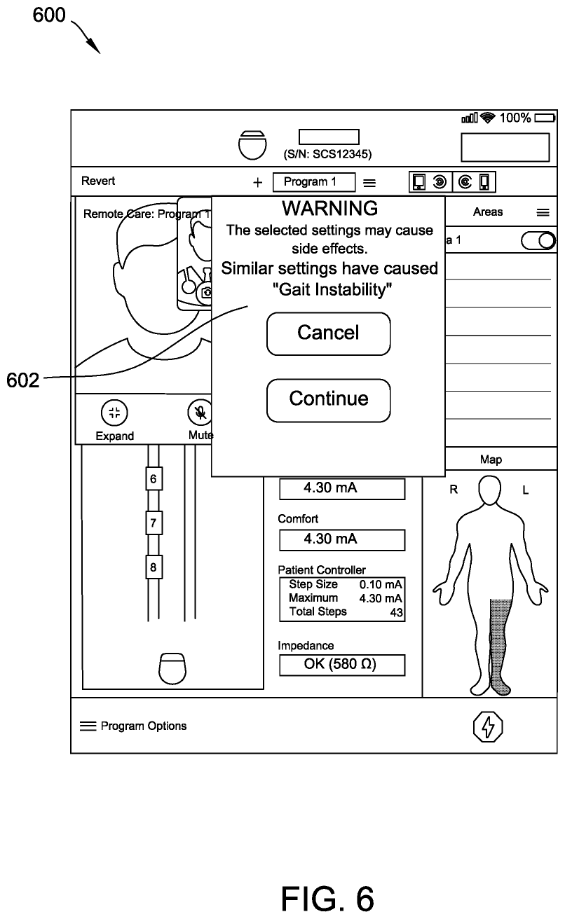

FIG. 6 shows another embodiment of a user interface 600 (e.g., to be displayed to clinician 138). User interface 600 enables a user (e.g., clinician 138) to modify settings of a neurostimulation system. Further, as shown in FIG. 6, user interface 600 displays a notification 602 triggered by a machine learning system trained using previous labels. In this example, notification 602 indicates potential side effects associated with the selected settings, and includes a previous label (“gait instability”) associated with such settings.

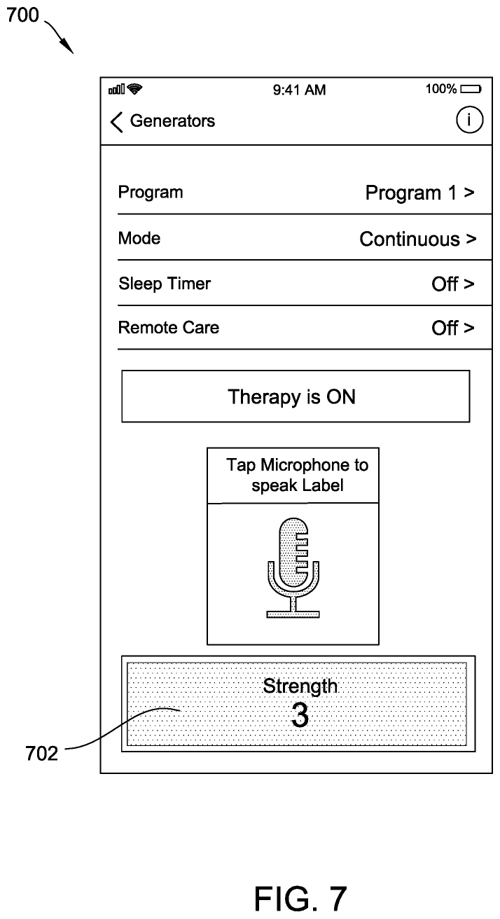

FIG. 7 shows another embodiment of a user interface 700 (e.g., to be displayed to patient 102). As shown in FIG. 7, the user (e.g., patient 102) has selected a programming setting 702 (e.g., by making a long-press selection on user interface 700), enabling the user to record a spoken label using a microphone on the device displaying user interface 700.

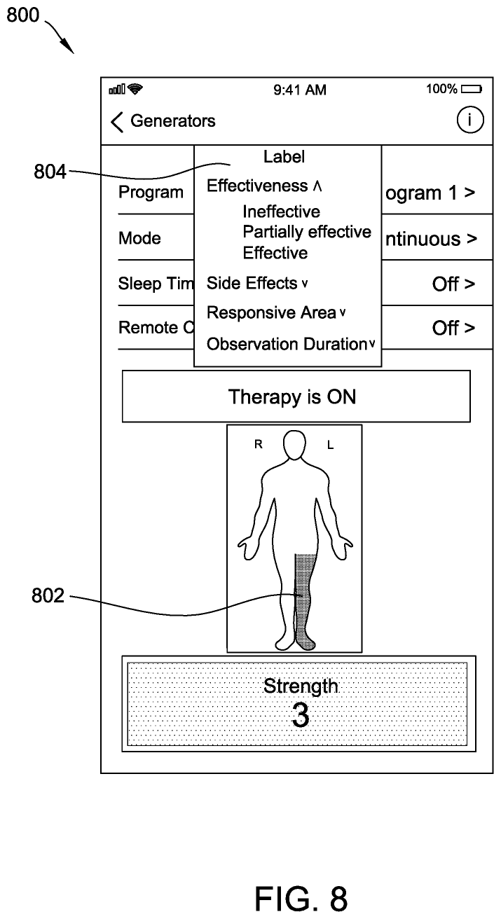

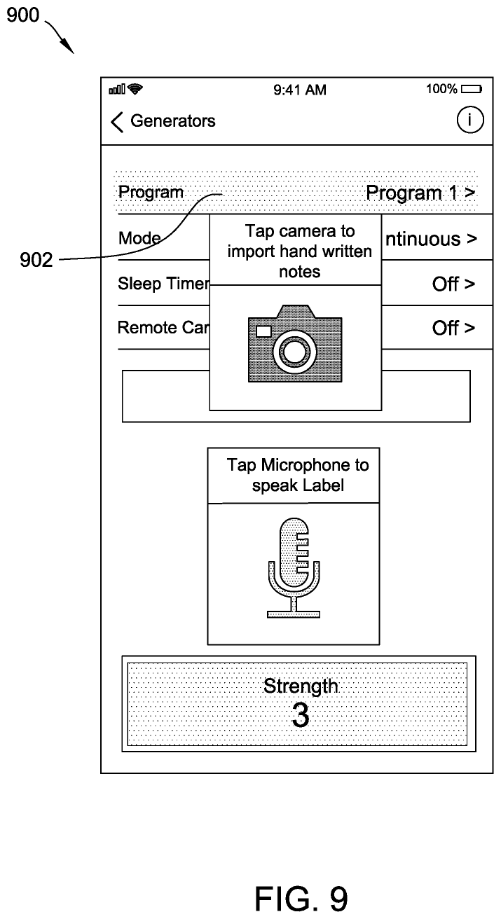

FIG. 8 shows another embodiment of a user interface 800 (e.g., to be displayed to patient 102). As shown in FIG. 8, the user (e.g., patient 102) has selected a map 802 of an affected body area (e.g., by making a long-press selection on user interface 800), enabling the user to add a label using a drop-down menu 804 including a nested tree of label options.

FIG. 9 shows another embodiment of a user interface 900 (e.g., to be displayed to patient 102). As shown in FIG. 9, the user (e.g., patient 102) has selected a programming setting 902 (e.g., by making a long-press selection on user interface 900), enabling the user to record a video clip of symptoms using a camera on the device displaying user interface 900.

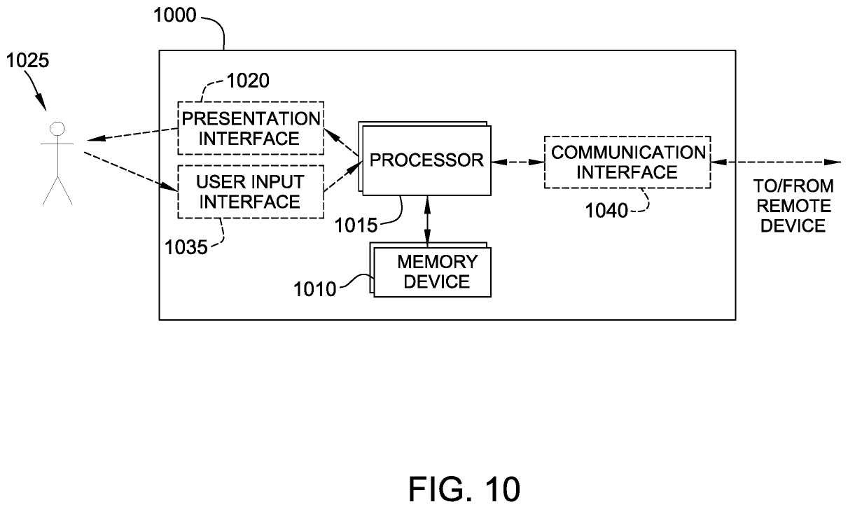

FIG. 10 illustrates one embodiment of a computing device 1000 that may be used to implement the systems and methods described herein. For example, computing device 1000 may be used to implement patient device 104 and/or clinician device 130 (both shown in FIG. 1).

Computing device 1000 includes at least one memory device 1010 and a processor 1015 that is coupled to memory device 1010 for executing instructions. In some embodiments, executable instructions are stored in memory device 1010. In this embodiment, computing device 1000 performs one or more operations described herein by programming processor 1015. For example, processor 1015 may be programmed by encoding an operation as one or more executable instructions and by providing the executable instructions in memory device 1010.

Processor 1015 may include one or more processing units (e.g., in a multi-core configuration). Further, processor 1015 may be implemented using one or more heterogeneous processor systems in which a main processor is present with secondary processors on a single chip. In another illustrative example, processor 1015 may be a symmetric multi-processor system containing multiple processors of the same type. Further, processor 1015 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein. In one embodiment, processor 1015 is a GPU (as opposed to a central processing unit (CPU)). Alternatively, processor 1015 may be any processing device capable of implementing the systems and methods described herein.

In this embodiment, memory device 1010 is one or more devices that enable information such as executable instructions and/or other data to be stored and retrieved. Memory device 1010 may include one or more computer readable media, such as, without limitation, dynamic random access memory (DRAM), static random access memory (SRAM), a solid state disk, and/or a hard disk. Memory device 1010 may be configured to store, without limitation, application source code, application object code, source code portions of interest, object code portions of interest, configuration data, execution events and/or any other type of data. In one embodiment, memory device 1010 is a GPU memory unit. Alternatively, memory device 1010 may be any storage device capable of implementing the systems and methods described herein.

In this embodiment, computing device 1000 includes a presentation interface 1020 that is coupled to processor 1015. Presentation interface 1020 presents information to a user 1025 (e.g., patient 102 or clinician 138). For example, presentation interface 1020 may include a display adapter (not shown) that may be coupled to a display device, such as a cathode ray tube (CRT), a liquid crystal display (LCD), an organic LED (OLED) display, and/or an “electronic ink” display. In some embodiments, presentation interface 1020 includes one or more display devices.

In this embodiment, computing device 1000 includes a user input interface 1035. User input interface 1035 is coupled to processor 1015 and receives input from user 1025. User input interface 1035 may include, for example, a keyboard, a pointing device, a mouse, a stylus, a touch sensitive panel (e.g., a touch pad or a touch screen), a gyroscope, an accelerometer, a position detector, and/or an audio user input interface. A single component, such as a touch screen, may function as both a display device of presentation interface 1020 and user input interface 1035.

Computing device 1000, in this embodiment, includes a communication interface 1040 coupled to processor 1015. Communication interface 1040 communicates with one or more remote devices. To communicate with remote devices, communication interface 1040 may include, for example, a wired network adapter, a wireless network adapter, and/or a mobile telecommunications adapter.

The embodiments described herein provide systems and methods for labeling data in an active implantable medical device system. The method includes capturing data associated with a remote therapy session between a patient device and a clinician device, and prompting, via a user interface, a user to label the captured data in response to the user selecting, via the user interface, at least one of i) an image of a patient displayed on the user interface, ii) a programming setting displayed on the user interface, and iii) an affected body area displayed on the user interface. The method further includes receiving, in response to the prompting, via the user interface, a user input associated with the captured data, generating, based on the user input, a label associated with the captured data, and storing the generated label in association with the captured data.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A method for labeling data in an active implantable medical device system, the method comprising:
capturing data associated with a remote therapy session between a patient device and a clinician device;
prompting, via a user interface, a user to label the captured data in response to the user selecting, via the user interface, at least one of i) an image of a patient displayed on the user interface, ii) a programming setting displayed on the user interface, and iii) an affected body area displayed on the user interface;
receiving, in response to the prompting, via the user interface, a user input associated with the captured data;
generating, based on the user input, a label associated with the captured data; and
storing the generated label in association with the captured data.

2. The method of claim 1, wherein prompting a user comprises prompting a clinician by displaying a prompt on the clinician device.

3. The method of claim 1, wherein prompting a user comprises prompting a patient by displaying a prompt on the patient device.

4. The method of claim 1, wherein receiving a user input comprises receiving a free-form label entered by the user.

5. The method of claim 1, wherein receiving a user input comprises receiving a video captured by the user.

6. The method of claim 1, wherein receiving a user input comprises recording a label spoken by the user.

7. The method of claim 1, wherein receiving a user input comprises capturing a physical note generated by the user using a camera.

8. The method of claim 1, wherein receiving a user input comprises receiving a selection from a list of previously generated labels.

9. A computing device for labeling data in an active implantable medical device system, the computing device comprising:
a memory device; and
a processor communicatively coupled to the memory device, the processor configured to:
capture data associated with a remote therapy session between a patient device and a clinician device;
prompt, via a user interface, a user to label the captured data in response to the user selecting, via the user interface, at least one of i) an image of a patient displayed on the user interface, ii) a programming setting displayed on the user interface, and iii) an affected body area displayed on the user interface;
receive, in response to the prompting, via the user interface, a user input associated with the captured data;
generate, based on the user input, a label associated with the captured data; and
store the generated label in association with the captured data in the memory device.

10. The computing device of claim 9, wherein to prompt a user, the processor is configured to prompt a clinician by displaying a prompt on the clinician device.

11. The computing device of claim 9, wherein to prompt a user, the processor is configured to prompt a patient by displaying a prompt on the patient device.

12. The computing device of claim 9, wherein to receive a user input, the processor is configured to receive a video captured by the user.

13. The computing device of claim 9, wherein to receive a user input, the processor is configured to receive a free-form label entered by the user.

14. The computing device of claim 9, wherein to receive a user input, the processor is configured to record a label spoken by the user.

15. The computing device of claim 9, wherein to receive a user input, the processor is configured to capture a physical note generated by the user using a camera.

16. The computing device of claim 9, wherein to receive a user input, the processor is configured to receive a selection from a list of previously generated labels.

17. Non-transitory computer-readable media having computer-executable instructions thereon, wherein when executed by a processor of a computing device, cause the processor of the computing device to:
capture data associated with a remote therapy session between a patient device and a clinician device;
prompt, via a user interface, a user to label the captured data in response to the user selecting, via the user interface, at least one of i) an image of a patient displayed on the user interface, ii) a programming setting displayed on the user interface, and iii) an affected body area displayed on the user interface;
receive, in response to the prompting, via the user interface, a user input associated with the captured data;
generate, based on the user input, a label associated with the captured data; and
store the generated label in association with the captured data.

18. The non-transitory computer-readable media of claim 17, wherein to prompt a user, the instructions cause the processor to prompt a clinician by displaying a prompt on the clinician device.

19. The non-transitory computer-readable media of claim 17, wherein to prompt a user, the instructions cause the processor to prompt a patient by displaying a prompt on the patient device.

20. The non-transitory computer-readable media of claim 17, wherein to receive a user input, the processor is configured to receive a free-form label entered by the user.
